(19) United States
(12) Patent Application Publication
(10) Pub. No.: US 2015/0246931 A1
(43) Pub. Date: Sep. 3, 2015

(54) PROCESS FOR THE PREPARATION OF TAZOBACTAM

(71) Applicant: HOSPIRA, INC., Lake Forest, IL (US)

(72) Inventors: Senthikumar Udayampalayam Palanisamy, Sozhanganallur (IN); Mohan Singaravel, Sozhanganallur (IN); Sivasankaran Venugopal, Sozhanganallur (IN); Kannadhasan Rajamanickam, Sozhanganallur (IN); Sakthivel Anandan, Sozhanganallur (IN); Loganathan Velraj, Sozhanganallur (IN)

(73) Assignee: HOSPIRA, INC., Lake Forest, IL (US)

(21) Appl. No.: 14/426,213

(22) PCT Filed: Sep. 5, 2013
(86) PCT No.: PCT/IB2013/058306

§ 371 Date: Mar. 5, 2015

(30) Foreign Application Priority Data
Sep. 6, 2012 (IN) 3690CHE2012

(51) Int. Cl. C07D 499/86

(52) U.S. Cl. CPC C07D 499/86

(57) ABSTRACT

The present invention relates to an improved process for the preparation of Tazobactam of formula (I) having reduced content of cresol.

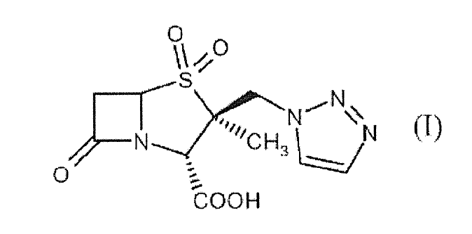

### FIELD

The present invention relates to an improved process for the preparation of Tazobactam of formula (I).

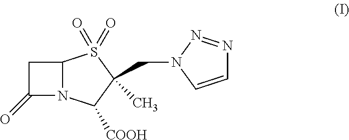 [SMILES: C[C@]1(Cn2ccnn2)[C@H](C(=O)O)N2C(=O)CC2S1(=O)=O]

### BACKGROUND

Tazobactam is chemically known as 2α-methyl-2β-(1,2,3-triazol-1-yl)-methylpenam-3α-carboxylate-1,1-dioxide and has a very low antibacterial activity. On the other hand, it exhibits a beta-lactamase inhibitory activity when irreversibly bonded to beta-lactamases produced by microorganisms. For this reason, Tazobactam may be used in combination with known antibiotics prone to be inactivated by beta-lactamases to allow them to exhibit their inherent antibacterial activity against beta-lactamase producing microorganisms. Tazobactam as a product is disclosed in U.S. Pat. No. 4,562,073.

Considering the importance of Tazobactam there are several literatures available which disclose various processes for the preparation of Tazobactam, some of which are described below.

U.S. Pat. No. 4,562,073 provides Tazobactam of formula (I) and its derivatives. This patent also describes a process for their preparation as shown in Scheme-1.

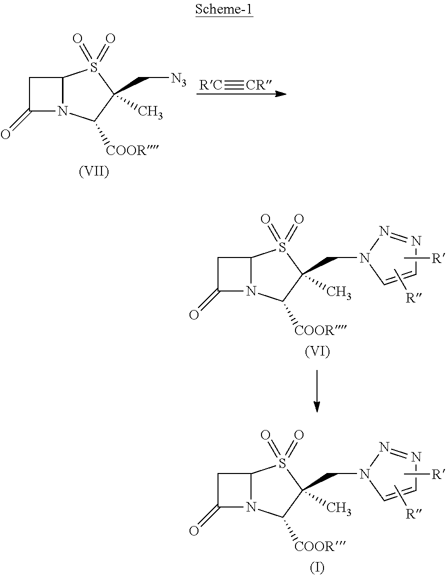

wherein R′ is hydrogen or trialkylsilyl; R″ is hydrogen, trialkylsilyl or COOM wherein M is hydrogen, C₁₋₁₈ alkyl, C₂₋₇ alkoxymethyl, etc., R′″ has the same meaning as M and R″″ represents carboxyl protecting group.

U.S. Pat. Nos. 4,891,369 and 4,933,444 disclose an approach, which involves the preparation of 2α-methyl-2β-triazolylmethylpenam derivative of formula (V)

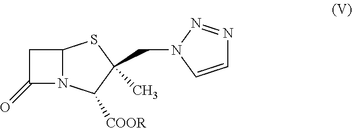

wherein R is a carboxy protecting group, by treatment of a β-halomethyl penam derivative of formula (IV), wherein X is chlorine or bromine and R is a carboxy protecting group, with 1,2,3-triazole.

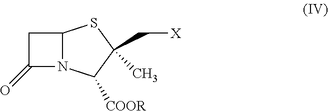

U.S. Pat. No. 4,507,239 provides a process which involves the preparation of 2α-methyl-2β-azidomethylpenam derivatives of formula (VII) by treatment of compound of formula (IV) with sodium azide in aqueous aprotic solvents.

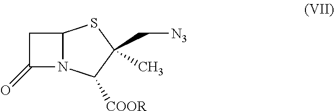

In yet another method disclosed in U.S. Pat. No. 4,895,941, penam sulfoxide of formula,

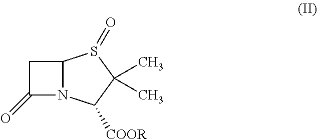

wherein R represents a carboxy protecting group, is treated with 2-trimethylsilyl-1,2,3-triazole in a sealed tube at elevated temperatures to give a mixture which upon column chromatography purification yields 2α-methyl-2β-triazolylmethyl penam derivative of formula (V).

U.S. Pat. No. 4,518,533 provides a process for the preparation of intermediate of formula (III)

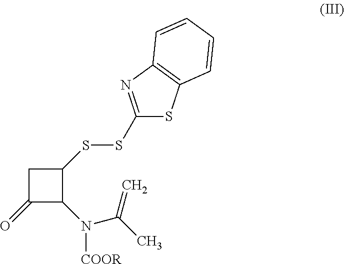

wherein the ester of penicillanic acid-1-oxide [compound of formula (II)] is reacted with 2-mercaptobenzothiazole in aliphatic hydrocarbon or aromatic hydrocarbon followed by isolation using column chromatographic method.

U.S. Pat. No. 7,273,935 provides a process for the preparation of compound of formula (VIII) by reacting compound of formula (III) with cyclizing agents like HCl or HBr and sodium nitrite.

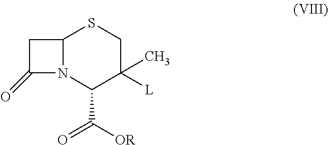

wherein R is carboxyl protecting group and L is a leaving group like Cl or Br.

U.S. Pat. No. 6,936,711 provides a process for the preparation of protected tazobactam [compound of formula (VI)] by reacting compound of formula (VIII) with 1,2,3-triazole using a base.

In addition, US patent namely U.S. Pat. No. 6,660,855, U.S. Pat. No. 7,692,003, and U.S. Pat. No. 7,547,777 claim process for the preparation of crystalline intermediates useful in the preparation of Tazobactam.

In general, dc-protection of p-nitrobenzyl/diphenylmethyl group in penem/penicillin core like Meropenem, Imipenem, Doripenem, Ertapenem, Faropenem, tazobactam and the like utilizes 1-10% of palladium on carbon, like commercially available 1.0%, 2.5%, 5.0%, 7.5% or 10%, which requires high pressure reactor.

U.S. Pat. No. 4,925,934 provides a de-protection method for 2α-methyl-2β-triazolylmethylpenam derivative of formula (VI) by reaction with m-cresol

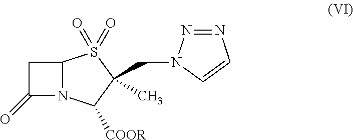

wherein R is selected from p-methoxybenzyl, diphenylmethyl(benzhydryl), tirmethoxybenzyl, 2,4-dimethoxybenzyl, 3,5-dimethoxy-4-hydroxybenzyl, 2,4,6-trimethylbenzyl, ditolylmethyl, dianisylmethyl or tert-butyl. The isolated product contains higher amount of m-cresol as an impurity.

U.S. Pat. No. 7,674,898 provides a process for the isolation of tazobactam by heating the aqueous solution containing Tazobactam before adjusting the pH. Before adjusting the pH of the aqueous solution containing tazobactam, the said solution was treated with ion-exchange resin column to purify the product. The use of ion-exchange resin and eluting the product is cumbersome on commercial scale.

Considering the importance of Tazobactam in healthcare treatment, the present inventors diligently worked to identify a robust and high yield process for the preparation of Tazobactam having cresol content below 5 ppm. A further purpose of the invention is to provide a manufacturing method that yields Tazobactam and its related intermediates with high purity and productivity.

### OBJECTIVE

An objective of the present invention is related to an improved process for the preparation of Tazobactam of formula (I) having pharmaceutically acceptable level of cresol.

Another objective of the present invention is to provide a simple and commercially viable process for the preparation of Tazobactam and its intermediates with improved productivity and purity.

### SUMMARY

Accordingly, the present invention provides an improved process for the preparation of Tazobactam of the formula (I)

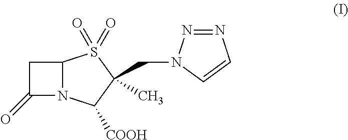 [SMILES: C[C@]1(Cn2ccnn2)[C@H](C(=O)O)N2C(=O)CC2S1(=O)=O]

which comprises the steps of:
i) reacting compound of formula (II) with mercaptan in the presence of an organic solvent to get compound of formula (III);
ii) converting the compound of formula (III) to compound of formula (IV) in the presence or absence of second organic solvent and phase transfer catalyst;
iii) converting the compound of formula (IV) to compound of formula (V);
iv) oxidizing compound of formula (V) to get compound of formula (VI);
v) deprotecting compound of formula (VI) with cresol to get tazobactam of formula (I); and
vi) extracting the Tazobactam of formula (I) into aqueous layer optionally using base; and
vii) acidifying the solution to yield Tazobactam of formula (I); wherein the improvement consists of either or both of the following
   a. carrying out steps (i) to (iii) in situ and without isolating the intermediates (III) and (IV);
   b. acidification in step (vii) is carried out in the presence of water-miscible solvent.
      The said process is shown in the following Scheme:

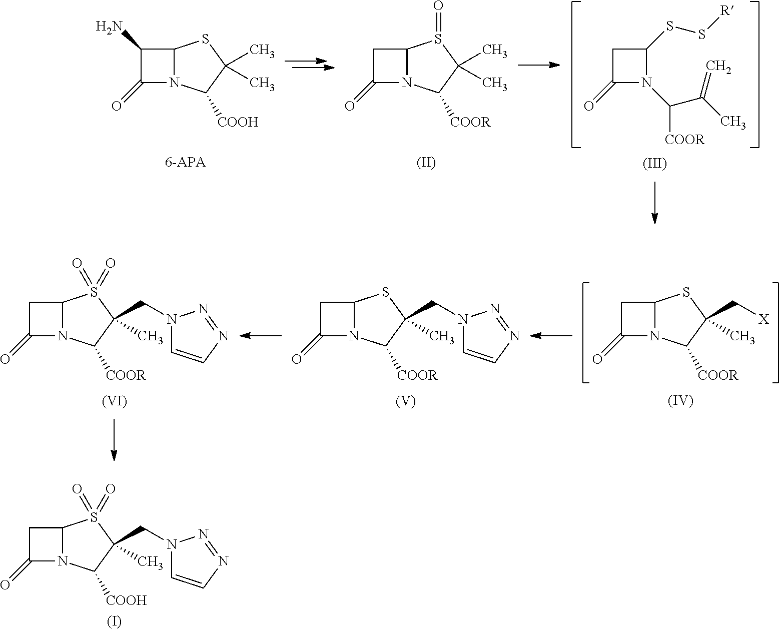

wherein R is selected from p-methoxybenzyl, diphenylmethyl(benzhydryl), 3,4,5-tirmethoxybenzyl, 2,4-dimethoxybenzyl, 3,5-dimethoxy-4-hydroxybenzyl, 2,4,6-trimethylbenzyl, ditolylmethyl, dianisylmethyl or tert-butyl; R′ is selected from benzothiazol-2-yl, benzoxazol-2-yl, benzimidazol-2-yl, 5-methyltetrazol-2-yl and X represents leaving groups selected from Cl, Br, I, methanesulfonyloxy, p-toluenesulfonyloxy, benzenesulfonyloxy or trifluoromethanesulfonyloxy.

Accordingly there is provided a process for the preparation of Tazobactam containing less than 5 ppm of cresol, the said process comprises the steps of:
A) deprotecting compound, of formula (VI)

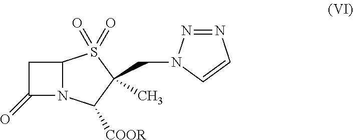

wherein R represents diphenylmethyl, p-methoxybenzyl, 3,4,5-tirmethoxybenzyl, 2,4-dimethoxybenzyl, 3,5-dimethoxy-4-hydroxybenzyl, 2,4,6-trimethylbenzyl, ditolylmethyl, dianisylmethyl or tort-butyl using cresol;
B) extracting the compound of formula (I) into aqueous layer optionally using base;
C) mixing water-miscible solvent with aqueous layer of step (II); and
D) acidifying the solution to a pH of below 3.0 to yield Tazobactam of formula (I).

### DETAILED DESCRIPTION

In an embodiment of the present invention, the mercaptan used in step (i) is selected from 2-mercaptobenzothiazole, 2-mercaptobenzoxazole, 2-mercaptobenzimidazole, 2-mercapto-5-methyltetrazole and organic solvent used in step (i) is selected from aromatic or aliphatic hydrocarbon like benzene, toluene, xylenes, hexanes, n-heptane, cyclohexane, cycloheptane and the like or mixtures thereof. The said reaction is preferably carried out at a temperature in the range of 50° C. to reflux temperature of the solvent used and under nitrogen atmosphere.

In another embodiment of the present invention, the compound of formula (III) is converted to compound of formula (V) in situ in the same solvent or by the addition of second organic solvent. The said reaction is preferably carried out at a temperature in the range of −25° C. to 50° C. temperature, preferably low temperature. A second organic solvent is selected from acetone, methyl ethyl ketone, methyl isobutyl ketone, acetonitrile, tetrahydrofuran, 2-methyl tetrahydrofuran, diethyl ether, isopropyl ether, methyl tort-butyl ether, dichloromethane, dichloroethane, chloroform, ethyl acetate, methyl acetate, n-butyl acetate, 1,4-dioxane or water can be added to facilitate the reaction.

In yet another embodiment of the present invention, the conversion of the compound of formula (III) to compound of formula (IV) in step (ii) is carried out using a nitrosating agent selected from a group comprising of nitrosyl chloride, nitrosyl bromide, nitrosyl iodide, nitrosyl methanesulfonate, nitrosyl benzenesulfonate, nitrosyl p-toluenesulfonate, nitrosyl trifluoromethane sulfonate and the like. The said nitrosating agents can be prepared by the reaction of inorganic or organic nitrite with hydrogen halide or sulfonic acid. The inorganic nitrite is selected from sodium nitrite, potassium nitrite, calcium nitrite and the organic nitrite is selected from amyl nitrite, isoamyl nitrite and the like. The hydrogen halide used is selected from hydrochloric acid, hydrobromic acid or hydroiodic acid and the sulfonic acid used is selected from methane sulfonic acid, benzene sulfonic acid, p-toluene sulfonic acid, trifluoromethane sulfonic acid and the like. To facilitate the reaction towards completion, a phase transfer catalyst (PTC) selected from a group comprising of quaternary ammonium cations, quaternary phosphonium cations or cyclic polyethers such as tetrabutylammonium bromide, benzyltrimethylammonium chloride benzyltributylammonium bromide, methyltrioctylammonium chloride benzyltrioctylammonium bromide, benzyltriphenylphosphonium bromide, 12-crown-4, 15-crown-5, 18-crown-6, dibenzo-18-crown-6 or diaza-18-crown-6 can be added. The said reaction is carried out at a temperature in the range of −20 to +5° C.; preferably −18 to −2° C. Instead of compound of formula (IV), the overall course of the reaction may proceed via compound of formula (VIII).

In still another embodiment of the present invention, the compound of formula (IV) is converted to compound of formula (V) in situ in the same solvent system. The reported prior art process isolates either compound of formula (III or IV) during the preparation of compound of formula (V) starting from compound of formula (II). Thus the present invention obviates the need of isolating the intermediates (compound of formula III & IV) thereby the overall productivity is increased in terms of reduced batch cycle time. In addition the use of second organic solvent and PTC helps for achieving complete conversion of the reaction and makes the process in situ. Accordingly the present invention provides a process for preparing Tazobactam comprising converting the compound of formula (II) into compound of formula (V) in situ and without isolating the compound of formula (III) and (IV) followed by converting the compound of formula (V) into Tazobactam.

In yet another embodiment of the present invention, the conversion of compound of formula (IV) to compound of formula (V) in step (iii) is carried out either by reacting the compound of formula (IV) with 1,2,3-triazole in the presence of base or by reacting the compound of formula (IV) with sodium azide followed by reacting the ensuing compound (VII) with acetylene. The said reaction can be conveniently carried out using the same solvent system or by addition of optional solvent like acetonitrile, tetrahydrofuran, water or mixtures thereof; preferably by the addition of minimum quantity of water; and phase transfer catalyst. Accordingly, the base employed for said conversion is selected from sodium carbonate, potassium carbonate, sodium bicarbonate, potassium bicarbonate, calcium carbonate, and anion exchange resin such as Diaion WA10, Diaion WA11, Diaion WA20, Diaion WA21, Diaion WA30, Amberlite-1RA67, Amberlite-IRA96SB, and Amberlite-LA2 Amberlite XE583 and Amberlite XT6050RF. As the resins disclosed in the present invention are only identified by their trade name, applicant herewith provided the links where the details of the resins can be accessed. http://www.diaion.com/en/products/ion_{—}04_{—}01.html for Diaion resins and http://www.dowwaterandprocess.com/en/products/ion_exchange_resins for Amberlite resins. The compound of formula (V) obtained is crystallized using solvents like ethanol, 1-propanol, isopropanol, 1-butanol, methyl isobutyl ketone, methyl tort-butyl ether, ethyl acetate, dichloromethane, isopropyl ether, heptane or mixtures thereof.

In one more embodiment of the present invention, the compound of formula (V) is converted to compound of formula (VI) in step (iv) in the presence of oxidizing agent selected from potassium permanganate, peracetic acid, trifluoroperacetic acid, m-chloroperbenzoic acid, magnesium monoperoxy phthalate, hydrogen peroxide, potassium peroxymonosulfate and the like in a solvent selected from acetone, water, acetic acid, dichloromethane and the like. After completion of the reaction, the reaction mass is quenched into water optionally containing hydrogen peroxide, sodium sulfite, sodium bisulfite, sodium metabisulfite or mixtures thereof (or vice versa). To the reaction mass, optionally water immiscible solvent (ethyl acetate, dichloromethane, ether and the like) is added and the product is isolated by conventional means such as extracting the product into solvent followed by distillation or distilling to crystallize the product in aqueous medium or by extracting the compound using hydrophobic solvents followed by distillation and crystallization using anti-solvent addition and the techniques known in prior arts.

In yet another embodiment of the present invention, the cresol used in the deprotection step (v) is selected from o-cresol, m-cresol or p-cresol, preferably m-cresol. The deprotection is carried out at a temperature in the range of 40° C. to 100° C. The base used for extraction of compound of formula (I) in step (vi) is selected from alkaline or alkaline earth metal carbonates, bicarbonates or hydroxides like sodium carbonate, sodium bicarbonate, potassium carbonate, potassium bicarbonate, calcium carbonate, sodium hydroxide or potassium hydroxide etc.

In another embodiment of the present invention, the water-miscible solvent used in step (b) is selected from a group comprising of methanol, ethanol, isopropyl alcohol, n-propanol, n-butanol, isobutanol, sec-butanol, tert-butanol, methoxyethanol, ethoxyethanol, acetone, tetrahydrofuran, 1,4-dioxane, acetonitrile etc, preferably methanol, ethanol. The term “water-miscible solvent” refers those solvent which forms a homogeneous solution when mixed with water.

In another embodiment of the present invention, the acidification in step (vii) is carried out using an acid selected from inorganic acids like hydrochloric acid, sulphuric acid or organic acids like formic acid, phosphoric acid, acetic acid etc. The pH is adjusted below 3.0, preferably below 2.0, more preferably below 1.0. Tazobactam obtained according to the prior art processes, for example as per the process reported in U.S. Pat. No. 4,925,934 has m-cresol at higher levels (more than 100 ppm). Considering the global regulatory scenario & hazard property of cresol (http://www.epa.gov/ttnatw01/hlthef/cresols.html) there is a need to produce Tazobactam with cresol content less than 2 ppm. The present invention provides a process for the preparation of Tazobactam which provides Tazobactam having less than 5 ppm of cresol, preferably less than 2 ppm, more preferably less than 1, ppm which comprises adjusting the pH of solution containing Tazobactam, wherein the pH of the solution is more than 4.0 and the said solution is obtained by de-protection of protected Tazobactam by using cresol, to pH less than 3.0 in the presence of water-miscible co-solvent. Thus the present invention provides an improved and robust process for the preparation of Tazobactam with cresol content below 5 ppm, preferably below 1 ppm. Further the present invention provides Tazobactam having m-cresol in the range of 0.01 ppm to 2 ppm, preferably Tazobactam with 0.01 ppm to 1 ppm, more preferably Tazobactam with 0.01 ppm to 0.75 ppm.

The starting material of formula (II) can be prepared by utilizing the processes reported in the prior art like (i) conversion of 6-APA to its 6-halo or 6,6-dihalo followed by dehalogenation, esterification and oxidation to get compound (II) (ii) conversion of 6-APA to its 6-halo or 6,6-dihalo analog followed by dehalogenation, oxidation, esterification to get compound (II) OR (iii) conversion of 6-APA to its 6-halo or 6,6-dihalo analog followed by oxidation, esterification and dehalogenation to get compound (II) OR (iv) conversion of 6-APA to compound (II) by reaction with alkali nitrite and hypophosphite followed by esterification and oxidation.

In the above processes, the oxidation can be carried out using hydrogen peroxide or peroxy acids in the presence or absence of catalysts. The dehalogenation can be carried out using zinc and acetic acid or ammonium salts like ammonium chloride or ammonium acetate or by using noble metal catalyst and hydrogen. The esterification can be carried out using a suitable reagent (benzophenone hydrazone or benzhydrol when R is diphenylmethyl). The solvents used in the dehalogenation step can be selected from ethyl acetate, acetone, tetrahydrofuran, 2-methyl tetrahydrofuran, methyl ethyl ketone, isobutyl ketone, isopropyl ether etc. The obtained compound (II) can be purified, if necessary by re-crystallization using solvents like acetone, methanol, toluene, ethyl acetate, tetrahydrofuran isopropyl ether, dichloromethane, n-butanol, isobutanol or mixtures thereof.

The present invention is exemplified by the following examples, which are provided for illustration only and should not be construed to limit the scope of the invention.

### Example 1

### Preparation of benzhydryl-3-methyl-7-oxo-3-(1H-1,2,3-triazol-1-yl methyl)-4-thia-1-azabicyclo[3.2.0]heptane-2-carboxylate (V)

In to toluene (1500 mL), benzhydryl-3,3-dimethyl-7-oxo-4-thia-1-azabicyclo[3.2.0]heptane-2-carboxylate-4-oxide [Compound (II)] (100 g), 2-mercaptobenzothiazole (42 g) were taken and refluxed till completion of the reaction. The toluene was partially recovered under vacuum followed by the addition of acetone and catalytic quantity of tetra-n-butylammonium bromide. The reaction mass was cooled followed by the addition of hydrobromic acid (99 g) and sodium nitrite (45 g) solution under stirring. The reaction mass was filtered and the filtrate was washed with water. The layers were separated and to the organic layer, 1,2,3-triazole (360 g) and calcium carbonate were added and stirred till completion of the reaction at around 10° C. The organic layer was washed with water. The organic layer was concentrated under vacuum followed by crystallization of benzhydryl-3-methyl-7-oxo-3-(1H-1,2,3-triazol-1-ylmethyl)-4-thia-1-azabicyclo[3.2.0]heptane-2-carboxylate using isopropyl alcohol.

Purity: 98.0% Assay: 98.4%

### Example 2

### Preparation of benzhydryl-3-methyl-7-oxo-3-(1H-1,2,3-triazol-1-ylmethyl)-4-thia-1-azabicyclo[3.2.0]heptane-2-carboxylate (V)

In to toluene (1500 mL), benzhydryl-3,3-dimethyl-7-oxo-4-thia-1-azabicyclo[3.2.0]heptane-2-carboxylate-4-oxide [Compound (II)] (100 g), 2-mercaptobenzothiazole (42 g) were taken and refluxed till completion of the reaction. The toluene was partially recovered under vacuum followed by the addition of acetonitrile and catalytic quantity of tetra-n-butylammonium bromide. The reaction mass was cooled followed by the addition of hydrobromic acid (99 g) and sodium nitrite (45 g) solution under stirring. The reaction mass was filtered and the filtrate was washed with water. The layers were separated and to the organic layer, 1,2,3-triazole (360 g) and anionic resin were added and stirred till completion of the reaction at low temperature. The resin was filtered and the separated organic layer was washed with water. The organic layer was concentrated under vacuum followed by crystallization of benzhydryl-3-methyl-7-oxo-3-(1H-1,2,3-triazol-1-ylmethyl)-4-thia-1-azabicyclo[3.2.0]heptane-2-carboxylate using isopropyl alcohol.

Yield: 50 g

Purity: 98.0% Assay: 98.4%

| S. No. | Reaction condition of step (ii) | Results |
|---|---|---|
| 1 | Reaction without PTC | Incomplete reaction |
| 2 | Reaction without second organic | Incomplete reaction |
| | solvent | |
| 3 | Reaction without PTC & second | Incomplete reaction |
| | organic solvent | |
| 4 | Example - 1& 2 | Reaction is very fast and |
| | | complete in 30 minutes |

### Advantages:

The above process obviates the isolation and crystallization of the intermediates (III) and (IV) and provides the compound (V) with purity and assay above 97%.
The prior art processes disclose the isolation of compound of formula (III) as residue by distilling the toluene layer completely. It was observed that there occurs an exothermic decomposition during the complete distillation. The present invention avoids the initiation of hazardous reaction and further the productivity is increased with reduced cycle time.
The prior art process of isolation of compound of formula (IV) has associated with storage problem, since the compound of formula (IV) degrades over a period of time.
The above table clearly indicates that the use of second organic solvent and PTC in step (ii) helps to achieve the complete conversion and avoid isolation of compound of formula (III) and (IV).

### Example 3

### Preparation of benzhydryl-3-methyl-7-oxo-3-(1H-1,2,3-triazol-1-ylmethyl)-4-thia-1-azabicyclo[3.2.0]heptane-2-carboxylate 4,4-dioxide (VI)

In to dichloromethane, benzhydryl-3-methyl-7-oxo-3-(1H-1,2,3-triazol-1-ylmethyl)-4-thia-1-azabicyclo[3.2.0]heptane-2-carboxylate (Compound V) (50 g) and acetic acid were added followed by the addition of potassium permanganate solution. The reaction mass was stirred till the completion of the reaction. A solution of sodium metabisulfite was added to the reaction mass and the product is isolated by conventional means.

Yield: 52 g

Purity: 99.17% Assay: 99.69%

### Example 4

### Preparation of Tazobactam (I)

Into m-cresol was added benzhydryl 3-methyl-7-oxo-3-(1H-1,2,3-triazol-1-ylmethyl)-4-thia-1-azabicyclo[3.2.0]heptane-2-carboxylate 4,4-dioxide (VI) (5 g) and heated at 50-55° C. till the completion of the reaction. The reaction mass was diluted with methyl isobutyl ketone. The reaction mass was extracted with sodium bicarbonate solution. The aqueous extract was acidified with hydrochloric acid to pH 3.0-4.0 and washed with methyl isobutyl ketone. Activated carbon was added, stirred and filtered. The filtrate was cooled to 0-5° C., and isopropyl alcohol (20 mL) was added followed by adjusting the pH to 1.0-2.0 using hydrochloric acid. The crystallized product was filtered, washed with water and dried.

Yield: 2.7 g

Purity: 99.9%

m-cresol content: 0.7 ppm

### Example 5

### Preparation of Tazobactam (I)

Into m-cresol was added benzhydryl 3-methyl-7-oxo-3-(1H-1,2,3-triazol-1-ylmethyl)-4-thia-1-azabicyclo[3.2.0]heptane-2-carboxylate 4,4-dioxide (VI) (5 g) and heated at 70-75° C. till the completion of the reaction. The reaction mass was diluted with dichloromethane. The reaction mass was extracted with potassium carbonate solution. The aqueous extract was acidified with hydrochloric acid to pH 3.0-4.0 and washed with dichloromethane. Activated carbon was added, stirred and filtered. To the filtrate, methanol (20 mL) was added followed by adjusting the pH to 1.0-2.0 using hydrochloric acid at 22-27° C. The crystallized product was filtered, washed with water and dried.

Yield: 2.6 g

Purity: 99.9%

m-cresol content: 0.24 ppm

### Example 6

### Preparation of Tazobactam (I)

Into m-cresol was added benzhydryl 3-methyl-7-oxo-3-(1H-1,2,3-triazol-1-ylmethyl)-4-thia-1-azabicyclo[3.2.0]heptane-2-carboxylate 4,4-dioxide (VI) (5 g) and heated at 60-65° C. till the completion of the reaction. The reaction mass was diluted with dichloromethane. The reaction mass was extracted with potassium carbonate solution. The aqueous extract was acidified with hydrochloric acid to pH 3.0-4.0 and washed with dichloromethane. Activated carbon was added, stirred and filtered. To the filtrate, ethanol (20 mL) was added followed by adjusting the pH to 1.0-2.0 using hydrochloric acid at 22-27° C. The crystallized product was filtered, washed with water and dried.

Yield: 2.6 g

Purity: 99.9%

m-cresol content: 0.31

### Reference Example-1

The process disclosed (example-1) in U.S. Pat. No. 4,925,934 was repeated to get Tazobactam

| Example | Co-solvent | m-cresol content |
|---|---|---|
| Reference example-1 | Nil | 167 ppm |
| Example - 4 | Isopropyl alcohol | 0.7 ppm |
| Example - 5 | Methanol | 0.24 ppm |
| Example - 6 | Ethanol | 0.31 ppm |

The above table clearly indicates that the use of water-miscible solvents helps to reduce the m-cresol content to less than 1 ppm.
The present process obviates the use of ion-exchange resin for the purification (Refer example-1 of U.S. Pat. No. 7,674,898) and provides a robust process for the industrial production of Tazobactam having less than 5 ppm, preferably less than 1 ppm.
   The m-cresol content in tazobactam acid is determined using HPLC with the following parameters

Colum: Zorbax SB C8 (150×4.6 mm, 3.50.

Mobile phase: Phosphate buffer: Acetonitile

Detector: UV at 200 nm

Column temperature: 30° C.

Flow rate: 0.8 mL/min

Run time: 15 min.

### Advantages

Some of advantages of the present invention include:
The present invention obviates the need of isolation of key inter mediates, thereby increasing the overall productivity.
As many steps are carried out in same solvent system and avoids the use of multiple solvent for extraction/anti-solvent usage, the present invention provides environment friendly and safe process for manufacturing Tazobactam.
Provides a robust process for the industrial production of Tazobactam having less than 1 ppm of cresol content.

1. An improved process for the preparation of Tazobactam of formula (I) 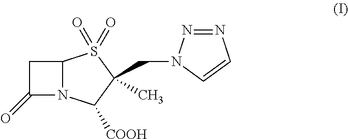 [SMILES: C[C@]1(Cn2ccnn2)[C@H](C(=O)O)N2C(=O)CC2S1(=O)=O]
which comprises the steps of:
i) reacting compound of formula (II) 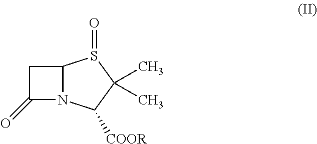
wherein R is selected from p-methoxybenzyl, diphenylmethyl(benzhydryl), 3,4,5-tirmethoxybenzyl, 2,4-dimethoxybenzyl, 3,5-dimethoxy-4-hydroxybenzyl, 2,4,6-trimethylbenzyl, ditolylmethyl, dianisylmethyl or tert-butyl with mercaptan of formula R′−SH in the presence of an organic solvent to get compound of formula (III); 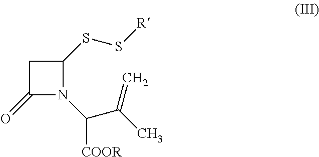
wherein R has the meaning given above and R′ is selected from benzothiazol-2-yl, benzoxazol-2-yl, benzimidazol-2-yl, 5-methyltetrazol-2-yl,
ii) converting the compound of formula (III) to compound of formula (IV) 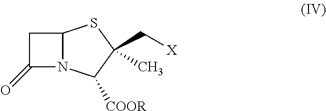
wherein R has the meaning given above and X represents a leaving group selected from the group consisting of chloro, bromo, iodo, mesylate, tosylate, besylate and triflate in the presence or absence of second organic solvent and phase transfer catalyst;
iii) converting the compound of formula (IV) to compound of formula (V); 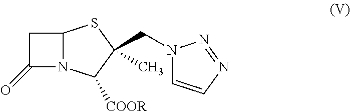
wherein R has the meaning given above;
iv) oxidizing compound of formula (V) to get compound of formula (VI); 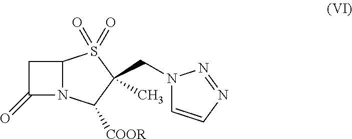
wherein R has the meaning given above;
v) deprotecting compound of formula (VI) with cresol to get tazobactam of formula (I); and
vi) extracting the tazobactam of formula (I) into aqueous layer optionally using base;
vii) acidifying the solution to yield Tazobactam of formula (I);
wherein the improvement consists of either or both of the following
a) carrying out steps (i) to (iii) in situ and without isolating the intermediates (III) and (IV);
b) acidification in step (vii) is carried out in the presence of water-miscible solvent.

2. A process for preparing Tazobactam comprising converting the compound of formula (II) into compound of formula (V) in situ and without isolating the compound of formula (III) and (IV) followed by converting the compound of formula (V) into Tazobactam.

3. The process according to claim 1, wherein the mercaptan used in step (i) is selected from a group comprising of 2-mercaptobenzothiazole, 2-mercaptobenzoxazole, 2-mercaptobenzimidazole or 2-mercapto-5-methyltetrazole and organic solvent used is selected from benzene, toluene, xylenes, hexanes, n-heptane, cyclohexane, cycloheptane or mixtures thereof.

4. The process according to claim 1, wherein the conversion in step (ii) is carried out using a reagent selected from a group comprising of nitrosyl chloride, nitrosyl bromide, nitrosyl iodide, nitrosyl methane sulfonate, nitrosyl benzenesulfonate, nitrosyl p-toluenesulfonate, nitrosyl trifluoromethanesulfonate optionally in the presence of an acid; the second organic solvent used in step (ii) is selected from acetone, methyl ethyl ketone, methyl isobutyl ketone, acetonitrile, tetrahydrofuran, 2-methyl tetrahydrofuran, diethyl ether, isopropyl ether, methyl tert-butyl ether, dichloromethane, dichloroethane, chloroform, ethyl acetate, methyl acetate, n-butyl acetate, 1,4-dioxane, water or mixtures thereof.

5. The process according to claim 1, wherein the phase transfer catalyst used in step (ii) is selected from a group comprising of tetrabutylammonium bromide, benzyltrimethylammonium chloride benzyltributylammonium bromide, methyltrioctylammonium chloride benzyltrioctylammonium bromide, benzyltriphenylphosphonium bromide, 12-crown-4, 15-crown-5, 18-crown-6, dibenzo-18-crown-6 or diaza-18-crown-6.

6. The process according to claim 1, wherein the conversion of compound of formula (IV) to compound of formula (V) in step (iii) is effected by reacting the compound of formula (IV) with 1,2,3-triazole in the presence of base selected from sodium carbonate, potassium carbonate, sodium bicarbonate, potassium bicarbonate, calcium carbonate, Diaion WA10, Diaion WA11, Diaion WA20, Diaion WA21, Diaion WA 30, Amberlite IRA67, Amberlite LA2, Amberlite IRA96SB, Amberlite XE583 and Amberlite XT6050RF or by conventional method.

7. The process as claimed in claim 1, wherein the oxidation in step (iv) is carried out using oxidising agents selected from a group comprising of potassium permanganate, peracetic acid, trifluoroperacetic acid, m-chloroperbenzoic acid, magnesium monoperoxy phthalate, hydrogen peroxide or potassium peroxymonosulfate.

8. The process as claimed in claim 1, wherein the cresol used in the deprotection in step (v) is selected from o-cresol, m-cresol or p-cresol and water-miscible solvent used in step (b) is selected from methanol, ethanol, isopropyl alcohol, n-propanol, n-butanol, isobutanol, seobutanol, tert-butanol, methoxyethanol, ethoxyethanol, acetone, tetrahydrofuran, 1,4-dioxane or acetonitrile.

9. The process as claimed in claim 1, wherein the tazobactam obtained is having less than 1 ppm of cresol.

10. An improved process for the preparation of Tazobactam of the formula (I) 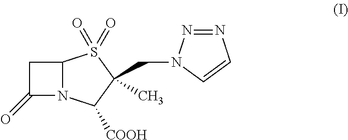 [SMILES: C[C@]1(Cn2ccnn2)[C@H](C(=O)O)N2C(=O)CC2S1(=O)=O]
which comprises the steps of:
I) deprotecting compound of formula (VI) 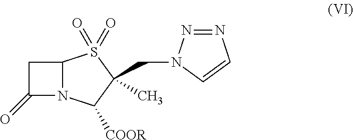
wherein R represents diphenylmethyl, p-methoxybenzyl, 3,4,5-tirmethoxybenzyl, 2,4-dimethoxybenzyl, 3,5-dimethoxy-4-hydroxybenzyl, 2,4,6-trimethylbenzyl, 1, ditolylmethyl, dianisylmethyl or tert-butyl using m-cresol;
II) extracting the compound of formula (I) into aqueous layer optionally using base;
III) mixing water-miscible solvent with aqueous layer of step (II);
IV) acidifying the aqueous layer from step (III) to a pH below 3.0 to yield Tazobactam of formula (I).

11. The process according to claim 10, wherein the base used in step (II) is selected from a group comprising of sodium carbonate, sodium bicarbonate, potassium carbonate, potassium bicarbonate, calcium carbonate, sodium hydroxide or potassium hydroxide.

12. The process according to claim 10, wherein the water-miscible solvent used in step (III) is selected from methanol, ethanol, isopropyl alcohol, n-propanol, n-butanol, isobutanol, seobutanol, tert-butanol, methoxyethanol, ethoxyethanol, acetone, tetrahydrofuran, 1,4-dioxane or acetonitrile.

13. The process according to claim 10, wherein the acidification in step (IV) is carried out using an acid selected from acetic acid, formic acid, phosphoric acid, hydrochloric acid or sulfuric acid.

14. Tazobactam having 0.01 ppm to 1 ppm of m-cresol.

15. (canceled)
